(19) United States
(12) Patent Application Publication
(10) Pub. No.: US 2003/0097151 A1
(43) Pub. Date: May 22, 2003

(54) Apparatus and mitochondrial treatment for glaucoma

(76) Inventors: Gregory T. Smedley, Irvine, CA (US); Hosheng Tu, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/281,873

(22) Filed: Oct. 25, 2002

Related U.S. Application Data
(60) Provisional application No. 60/352,026, filed on Oct. 25, 2001.

(51) Int. Cl. A61N 1/00

(52) U.S. Cl. 607/2

(57) ABSTRACT

A method is provided for treatment of glaucoma comprising stimulating mitochondria of ophthalmologic cells with energy effective for stimulating the mitochondria, wherein the energy source may be a physical source or biochemical source of monoamine oxidase inhibitors. A unique endoscope-microscope interface is disclosed which advantageously provides a simultaneous view of the microscope field of view and the endoscope field of view to the operator or surgeon.

### CROSS REFERENCE TO RELATED APPLICATIONS

This application claims the benefit of U.S. Provisional Application No. 60/352,026, filed Oct. 25, 2001, entitled “MICROSCOPE-EYEPIECE INTERFACE FOR ENDOSCOPE AND MITOCHONDRIAL TREATMENT FOR GLAUCOMA”, the entirety of which is hereby incorporated by reference herein.

### BACKGROUND OF THE INVENTION

1. Field of the Invention

The invention relates to general therapeutic procedures for treating glaucoma. More particularly, the invention relates to a treatment of glaucoma in combination with an ab interno procedure for maintaining the intraocular pressure by promoting intraocular liquid to flow out of an anterior chamber of the eye through a surgically stented pathway and/or a mitochondrial stimulating therapy for neural protection.

2. Description of the Related Art

As is well known in the art, a human eye is a specialized sensory organ capable of light reception and is able to receive visual images. Aqueous humor is a transparent liquid that fills the region between the cornea, at the front of the eye, and the lens. A trabecular meshwork, located in an anterior chamber angle formed between the iris and the cornea, serves as a drainage channel for intraocular liquid from the anterior chamber, which maintains a balanced pressure within the anterior chamber of the eye.

Historically, about two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucoma's, while the ultimate goal for glaucoma treatment is neural protection that will aid in the preservation of sight.

In glaucoma associated with an elevation in eye pressure (intraocular hypertension), the source of resistance to outflow is mainly in the trabecular meshwork. The tissue of the trabecular meshwork allows the aqueous humor (herein also referred to as “aqueous” that is one component of the “intraocular liquid” referred to herein) to enter Schlemm's canal, which then empties into aqueous collector channels in the posterior wall of Schlemm's canal and then into aqueous veins, which form the episcleral venous system. Aqueous is continuously secreted by a ciliary body around the lens, so there is a constant flow of aqueous from the ciliary body to the anterior chamber of the eye.

Pressure within the eye is determined by a balance between the production of aqueous and its exit through the trabecular meshwork (major route) and uveal scleral outflow (minor route). The portion of the trabecular meshwork adjacent to Schlemm's canal (the juxtacanilicular meshwork) causes most of the resistance to aqueous outflow.

### SUMMARY OF THE INVENTION

Because the trabecular meshwork and juxtacanilicular tissue together provide the majority of resistance to the outflow of aqueous, they are logical targets for surgical channeling with a stented pathway for maintaining balanced intraocular pressure. In some glaucoma patients, this surgical channeling becomes the only feasible alternative for lowering the intraocular pressure because of the patient's intolerance to glaucoma medicine.

The other therapeutic treatment for glaucoma is to lessen apoptotic degradation of optic nerve cells by energizing the mitochondria. A mitochondria stimulating drug may be incorporated onto or within a stent implant for drug slow release to some target cells in an eye.

Lynch et al. in U.S. Pat. No. 6,450,984, the entire contents of which are hereby incorporated by reference herein, disclose a glaucoma shunt implant providing an aqueous passageway from an anterior chamber to Schlemm's canal, wherein the implant lies within the trabecular meshwork of the eye.

It is one object of the invention to provide a mitochondria stimulating drug incorporated onto an implant for drug slow release to some target cells of the trabecular meshwork or the posterior chamber in an eye.

Many types of open angle glaucoma exist; therefore, a number of potential therapeutic mitochondrial interventions may be possible. One primary aspect of this therapy is the stimulation of mitochondrial survival/function to prevent demise and secondary apoptosis (programmed cell death).

In primary open angle glaucoma (POAG), the intraocular pressure (IOP) increases in response to a decrease in the outflow of aqueous. Research has shown that the number of juxtacanalicular endothelial cells in Schlemm's canal is lower in individuals with POAG compared to normals (Grierson et al., Exp Eye Res, 1984;39(4):505-512). Since these cells are involved in the energy-dependent egress of aqueous, their demise results in elevated IOP. Therefore, the mitochondrial treatment objectives for POAG include not only the prevention of further endothelial cell death, but also the restoration or boosting of mitochondrial function in the remaining cells.

In one aspect, the target tissue is in the anterior chamber; therefore, this therapeutic arm may allow the use of topical therapy. The visual loss that results from elevated IOP is caused by the death of retinal ganglion cells and the loss of nerve fiber layer (NFL) in the retina. This death may be secondary to decreased nutrition (or decreased tropic factors) caused by the pressure-induced reduction of retrograde axioplasmic transport. These cells may be made more resilient to elevated IOP with mitochondrial stimulating therapy; however, systemic drug delivery may be required to effectively dose them.

In another aspect, the drug slow release therapy to target tissue may allow the use of a drug-coated implant in an eye. The loss of retinal ganglion cells and nerve fiber layer in normal tension glaucoma (NTG) is similar, but without the elevation in IOP; therefore, the treatment will likely be based on a similar mitochondrial stimulating therapy. The gradual loss of visual function in NTG individuals is similar to that seen in individuals with advanced POAG and controlled IOP.

Tatton in U.S. Pat. No. 5,981,598, the entire contents of which are hereby incorporated by reference herein, discloses a method for administering a therapeutically effective amount of a deprenyl compound to a subject such that the subject is treated for glaucoma.

It is one object of the invention to provide a method for stimulating mitochondria so as to mitigate apoptotic degradation of optic nerve cells for neural protection. More particularly, such a mitochondria stimulating drug is incorporated onto an implant for drug slow release in an eye.

Ghosh et al. in U.S. Pat. No. 6,268,398, the entire contents of which are hereby incorporated by reference herein, disclose compounds for treating mitochondria-associated diseases with functions of mitochondria protecting, anti-apoptotic or pro-apoptotic.

It is one object of the invention to provide a method for stimulating mitochondria so as to mitigate apoptotic degradation of optic nerve cells for neural protection. More particularly, such a mitochondria stimulating drug is incorporated onto an implant for drug slow release in an eye.

What is needed or desirable, therefore, is a procedure for either an ab interno trabecular stenting for aqueous drainage to maintain substantially balanced intraocular pressure or providing mitochondrial stimulating therapy for treating glaucoma or optical nerve degeneration.

A method is provided for treatment of glaucoma comprising stimulating mitochondria of ophthalmologic cells with energy effective for stimulating the mitochondria, wherein the energy source may be a physical source or biochemical source of monoamine oxidase inhibitors. A unique endoscope-microscope interface is disclosed which advantageously provides a simultaneous view of the microscope field of view and the endoscope field of view to the operator or surgeon.

Some embodiments of the invention relate to a method of treating mitochondria in a cell of a glaucoma patient comprising stimulating mitochondria of the cell with an energy source sufficient to increase cellular energy production.

It is one object of the invention to provide a method of treating glaucoma comprising stimulating mitochondria of ophthalmologic cells with a physical or biochemical energy effective for stimulating the mitochondria of the cells.

In one aspect of the invention, the physical energy may be selected from a group comprising ultrasonic energy, microwave energy, optical light energy, laser energy, electromagnetic energy, and/or combinations thereof, wherein the mode of delivering energy is selected from a group comprising continuous, intermittent, programmed, and/or combinations thereof.

In another aspect of the invention, the biochemical energy is provided by a mitochondrial stimulating agent, wherein the mitochondrial stimulating agent may be a monoamine oxidase inhibitor, preferably comprising deprenyl compounds.

For purposes of summarizing the invention, certain aspects, advantages and novel features of the invention have been described herein above. Of course, it is to be understood that not necessarily all such advantages may be achieved in accordance with any particular embodiment of the invention. Thus, the invention may be embodied or carried out in a manner that achieves or optimizes one advantage or group of advantages as taught or suggested herein without necessarily achieving other advantages as may be taught or suggested herein.

All of these embodiments are intended to be within the scope of the invention herein disclosed. These and other embodiments of the invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Having thus summarized the general nature of the invention and some of its features and advantages, certain preferred embodiments and modifications thereof will become apparent to those skilled in the art from the detailed description herein having reference to the figures that follow, of which:

FIG. 1 is a coronal, cross section view of an eye.

FIG. 2 is an enlarged cross section view of an anterior chamber angle of the eye of FIG. 1.

FIG. 3 is front elevation view of a stent implant having features and advantages in accordance with one embodiment of the invention.

FIG. 4 is a top plan view of the stent implant of FIG. 3 along line 4-4 of FIG. 3.

FIG. 5 is a bottom end view of the stent implant of FIG. 3 along line 5-5 of FIG. 3.

FIG. 6 is a simplified schematic illustration of the stent implant of FIG. 3 implanted within the eye having features and advantages in accordance with one embodiment of the invention.

FIG. 7 illustrates one preferred exemplary method for placing a stent implant at a desired implant site and having features and advantages in accordance with one embodiment of the invention.

FIG. 8 illustrates one preferred exemplary method of using a stent device for establishing an outflow pathway in an eye and having features and advantages in accordance with one embodiment of the invention.

FIG. 9 is a schematic illustration of an endoscope image as viewed by one eye and a microscope image as viewed by the other eye through a stereomicroscope and endoscope assembly having features and advantages in accordance with one embodiment of the invention.

FIG. 10 is a schematic illustration of an endoscope image adjacent a microscope image as viewed through a stereomicroscope and endoscope assembly having features and advantages in accordance with one embodiment of the invention.

FIG. 11 is a schematic illustration of an endoscope image overlaid on a microscope image as viewed through a stereomicroscope and endoscope assembly having features and advantages in accordance with one embodiment of the invention.

FIG. 12 is a schematic illustration of an endoscope image adjacent a microscope image as viewed through a monocular microscope and endoscope assembly having features and advantages in accordance with one embodiment of the invention.

FIG. 13 is a schematic illustration of an endoscope image overlaid on a microscope image as viewed through a monocular microscope and endoscope assembly having features and advantages in accordance with one embodiment of the invention.

FIG. 14 is a simplified view of an optical assembly comprising an eyepiece and endoscope interface having features and advantages in accordance with one embodiment of the invention.

FIG. 15 is a simplified detail view of the interconnection between the eyepiece and the endoscope of FIG. 14 and having features and advantages in accordance with one embodiment of the invention.

FIG. 16 is a simplified view of a stereomicroscope assembly including the optical assembly of FIG. 14 and having features and advantages in accordance with one embodiment of the invention.

FIG. 17 is a simplified view of a monocular microscope assembly including the optical assembly of FIG. 14 and having features and advantages in accordance with one embodiment of the invention.

FIG. 18 is a simplified view of a stereomicroscope and endoscope assembly having features and advantages in accordance with one embodiment of the invention.

FIG. 19 is a simplified view of a monocular microscope and endoscope assembly having features and advantages in accordance with one embodiment of the invention.

FIG. 20 is a schematic illustration of drug release from a coating on an implant having features and advantages in accordance with one embodiment of the invention.

FIG. 21 is a schematic illustration of drug release from within an implant having features and advantages in accordance with one embodiment of the invention.

### DETAILED DESCRIPTION OF THE PREFERRED EMBODIMENTS

The drawings generally illustrate devices and methods related to the treatment of glaucoma. Some preferred embodiments of the invention described herein and/or below relate particularly to a therapeutic treatment of glaucoma in a surgical treatment of glaucoma through maintaining normal intraocular pressure and/or stimulating trabecular meshwork function.

While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described herein and/or below.

The function of the aqueous production and transmission depends on the physiochemical state of the tissue in an anterior chamber and along the aqueous outflow channels. These proteins of the tissue, like the proteins of other organs, are sensitive to changes in the properties of their surrounding fluid. Changes in the concentration of dissolved salts, in the osmotic pressure, in the pH or in the enzyme activity of the surrounding fluid can alter the properties of the tissue proteins. Also, like other organs, changes to the proteins of the lens occur with age. Particularly the trabecular meshwork tissue contains mitochondria, which might affect the aqueous transmission characteristics therethrough.

Some aspects of the invention provide a method of treating glaucoma of an eye while maintaining mitochondrial function of the trabecular meshwork or the aqueous outflow channels system. The method generally comprising steps of establishing an opening through trabecular meshwork, implanting a trabecular stent having a lumen therein with optionally drug slow-releasing capability. The normal physiological intraocular pressure (IOP) is preferably maintained between about 10 mm Hg and about 21 mm Hg.

Other aspects of the invention provide an improved instrument for assisting the implantation of a trabecular stent for enhancing the aqueous flow through or bypassing an existing aqueous flow system.

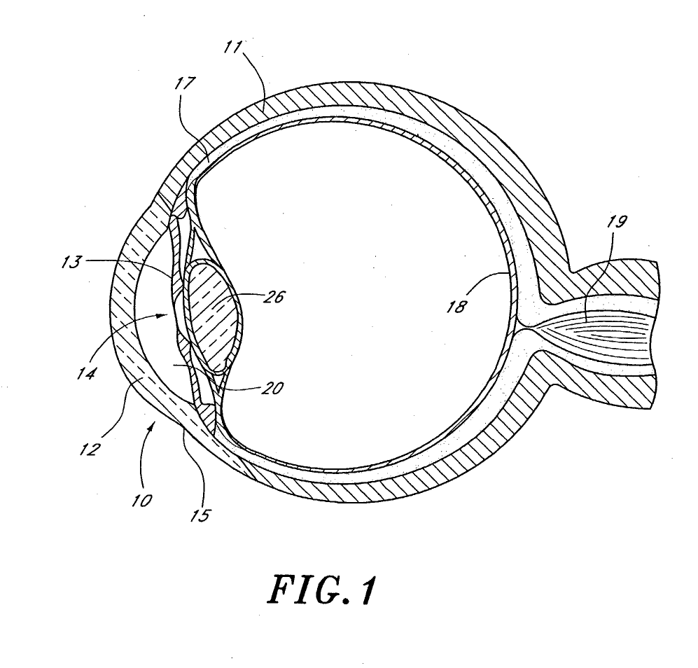

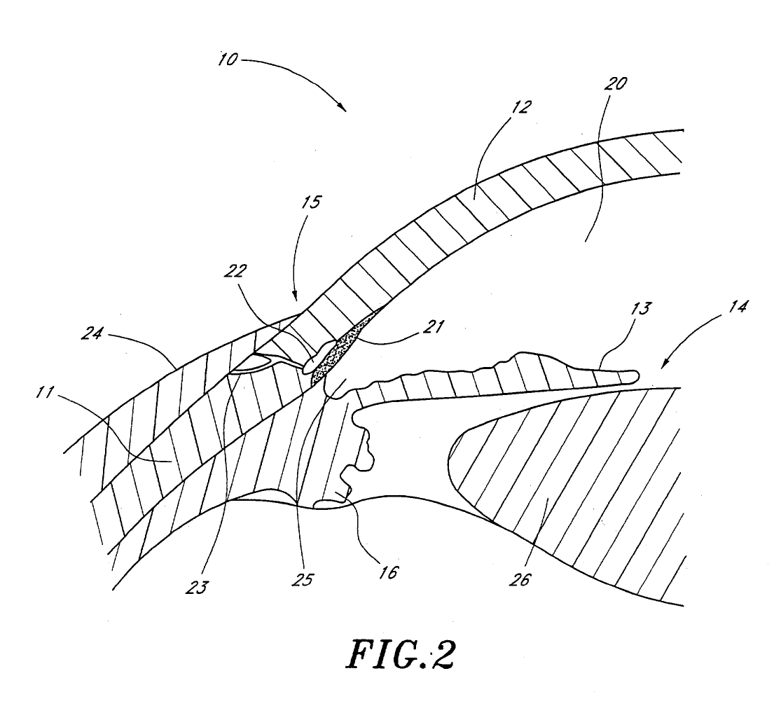

For background illustration purposes, FIG. 1 shows a sectional view of an eye 10, while FIG. 2 is a close-up view showing the relative anatomical locations of a trabecular meshwork 21, an anterior chamber 20, and Schlemm's canal 22. Thick collagenous tissue known as sclera 11 covers the entire eye 10 except that portion covered by a cornea 12. The cornea 12 is a thin transparent tissue that focuses and transmits light into the eye and through a pupil 14 which is a circular hole in the center of an iris 13 (colored portion of the eye). The cornea 12 merges into the sclera 11 at a juncture referred to as a limbus 15. A ciliary body 16 begins internally in the eye and extends along the interior of the sclera 11 and is coextensive with a choroid 17. The choroid 17 is a vascular layer of the eye, located between the sclera 11 and an underlying retina 18. An optic nerve 19 transmits visual information to the brain and is the anatomic structure that is progressively destroyed by glaucoma.

The anterior chamber 20 of the eye 10 (FIGS. 1 and 2), which is bound anteriorly by the cornea 12 and posteriorly by the iris 13 and a lens 26, is filled with aqueous humor (herein also referred to as “aqueous”). Aqueous is produced primarily by the ciliary body 16 and reaches an anterior chamber angle 25, formed between the iris 13 and the cornea 12, through the pupil 14.

Still referring in particular to FIGS. 1 and 2, in a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure (IOP) is maintained by an intricate balance between secretion and outflow of aqueous in the manner described above.

Glaucoma is, in most cases, characterized by an excessive buildup of aqueous in the anterior chamber 20 (FIGS. 1 and 2) which leads to an increase in intraocular pressure (IOP). Fluids are relatively incompressible, and thus intraocular pressure (IOP) is distributed relatively uniformly throughout the eye 10. The lens of the human eye 26 is a crystalline lens that comprises an outer capsule with anterior and posterior surfaces, the lens containing a clear central matrix.

As shown in FIG. 2, the trabecular meshwork 21 is adjacent a small portion of the sclera 11. Exterior to the sclera 11 is a conjunctiva 24. Traditional procedures that create a hole or opening for implanting a device through the tissues of the conjunctiva 24 and sclera 11 involve extensive surgery known as ab externo procedures, as compared to surgery for implanting a device, as described herein known as ab interno procedures, which ultimately resides entirely within the confines of the sclera 11 and cornea 12.

Some embodiments relate to a method for increasing aqueous humor outflow in an eye of a patient to reduce the intraocular pressure (IOP) therein. In certain embodiments, the method comprises bypassing diseased or deficient trabecular meshwork at the level of trabecular meshwork and thereby restoring existing outflow pathways. In other embodiments, the method comprises bypassing diseased trabecular meshwork at a level of the trabecular meshwork with a trabecular stent device and using existing outflow pathways.

Stent Implant

Various stent implants or devices may efficaciously be utilized in embodiments of the invention. Some of these stent implants are generally referred to by the reference numeral 31 herein and include the stent implants 31a, 31b and 31c disclosed herein.

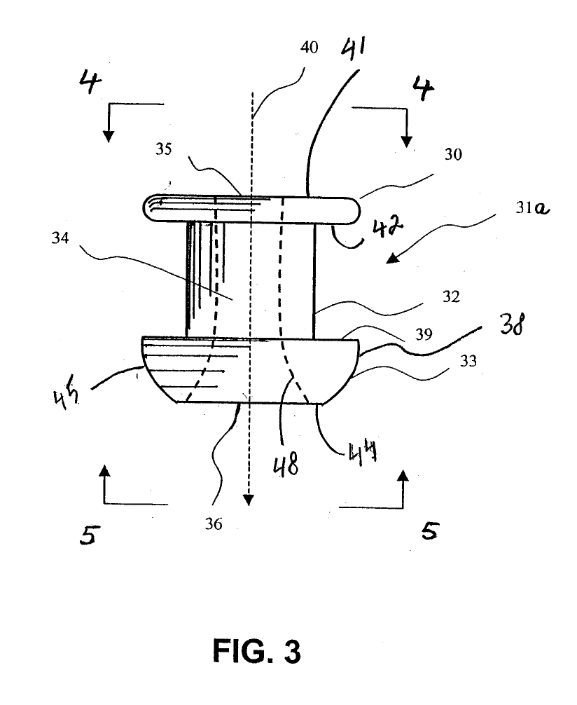

FIGS. 3-5 show different views of an opthalmological stent implant 31a constructed in accordance with one embodiment. FIG. 6 illustrates the implantation of the stent 31a within the eye 10. The stent implant 31a may comprise an elongated stent or other appropriate shape, size or configuration. In the illustrated embodiment, the stent implant 31a is in the form of an elongated tubular element and generally comprises an inlet or proximal section 30, an outlet or distal section 33, a medial section 32 therebetween and a lumen or passage 34 extending therethrough.

Referring in particular to FIGS. 3-6, and as best seen in FIG. 6, in use, the inlet section 30 is positioned in the anterior chamber 20 of the eye 10 at about an interior surface 46 of the trabecular meshwork 21 (or extending from the interior surface 46 into the anterior chamber 20) and the outlet end or the outlet section 33 is positioned at about an exterior surface 47 of the diseased trabecular meshwork 21. As illustrated in FIG. 6, the trabecular meshwork interior side or surface 46 faces the anterior chamber 20 and the trabecular meshwork exterior side or surface 47 faces Schlemm's canal 22.

In some embodiments, the stent outlet section or end may be positioned into fluid collection channels of the existing outflow pathways. In some embodiments, the existing outflow pathways may comprise Schlemm's canal 22, while the stent is preferably positioned inside Schlemm's canal 22 not necessarily circumferentially along the canal.

In some aspects, the stent 31a (FIGS. 3-6) is essentially held firmly by the trabecular meshwork 21 that is radially outwardly compressed by the middle section 32 of the stent body, rather than by the circumference of Schlemm's canal 22. The stent outlet section or end 33 may be further positioned into fluid collection channels up to the level of the aqueous veins 23 (see FIG. 2) with the stent 31a inserted within the eye 10. In general, the stent implant may be an axisymmetric stent or other configuration suitable for use with the methods taught or suggested herein.

In the illustrated embodiment of FIGS. 3-6, the proximal inlet section or portion 30 is generally in the form of a circular disc and has a proximal-most end or upper surface 41 and a lower surface 42. In modified embodiments, the stent proximal section may be shaped in other suitable manners with efficacy, as needed or desired, for example, oval, ellipsoidal, and the like. As best seen in FIG. 6, when the stent 31a is implanted within the eye 10, the upper surface 41 is exposed to or within the anterior chamber 20 while the lower surface 42 is seated on or abuts against the interior surface 46 of the trabecular meshwork 21 to stabilize the implanted stent 31.

In the illustrated embodiment of FIGS. 3-6, the medial or middle section or portion 32 is generally cylindrical in shape and has a generally circular cross-section. In modified embodiments, the stent medial section may be shaped in other suitable manners with efficacy, as needed or desired, for example, oval, ellipsoidal, and the like. As best seen in FIG. 6, when the stent 31a is implanted within the eye 10, the medial section 32 is received within an opening 103 within the trabecular meshwork 21. Preferably, the middle section 32 is configured and sized to fit the opened region 103 of the trabecular meshwork 21 and radially outwardly compress the trabecular meshwork 21 around the opening 103 to stabilize the stent 31a.

In the illustrated embodiment of FIGS. 3-6, the distal outlet section or portion 33 has an upper surface 39, a distal-most end or surface 44 and a tapered or curved outer surface 45 therebetween. The outer periphery of the outlet section 33 is generally circumferential or circular in shape. In modified embodiments, the stent distal section may be shaped in other suitable manners with efficacy, as needed or desired, for example, oval, ellipsoidal, and the like.

As best seen in FIG. 6, when the stent 31a is implanted within the eye 10, the distal section 33 is received within Schlemm's canal 22 and the upper surface 39 abuts against the exterior surface 47 of the trabecular meshwork 21 to stabilize the implanted stent 31a. The distal section 33 may have a bulged outlet end or protrusion 38 and/or other bulging or protruding retention device or mechanism for stabilizing the stent implant 31 inside the existing outflow pathways after implantation, for example, a barb, among others.

For stabilization purposes, the outer surface of the distal section 33 may comprise a stubbed surface, a ribbed surface, a surface with pillars, a textured surface, and the like, or a combination thereof. In some embodiments, the distal section 33 may be curved or bent at an angle with reference to the proximal section 30 and/or the medial section 32. For example, the stent implant my be substantially L-shaped or T-shaped with the proximal and/or medial sections comprising a snorkel portion extending through the trabecular meshwork 21 and the distal section extending within Schlemm's canal 22 and/or other aqueous outflow pathways. The angulations(s) may be substantially perpendicular, acute angled or obtuse angled, as needed or desired.

In the illustrated embodiment of FIGS. 3-6, the lumen 34 has an upper opening, orifice or port 35 at the proximal end 41 and a lower opening, orifice or port 36 at the distal end 44. The lumen 34 has a generally circumferential or circular cross-section with a tapered or curved surface 48 within the distal section 33. In modified embodiments, the stent lumen may be shaped in other suitable manners with efficacy, as needed or desired, for example, oval, ellipsoidal, and the like, or some other shape configured and adapted for effective aqueous entrance and transmission. In some embodiments, the stent implant 31a may have a plurality of lumens to facilitate multiple flow transportation, as needed or desired.

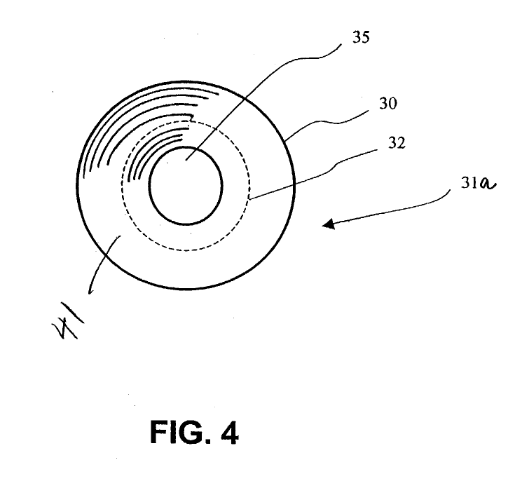

As best seen in FIG. 4, the lumen upper orifice 35 is generally circular or round in shape. In modified embodiments, the lumen upper orifice may be shaped in other suitable manners with efficacy, as needed or desired, for example, oval, ellipsoidal, and the like, or some other shape configured and adapted for effective aqueous entrance and transmission. The stent implant 31a may comprise one or more inlet openings 35 at the inlet section 30 to allow adequate outflow of aqueous, as needed or desired.

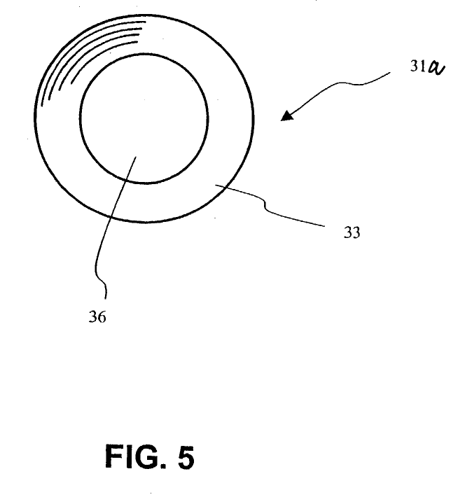

As best seen in FIG. 5, the lumen lower orifice 36 is generally circular or round in shape. In modified embodiments, the lumen lower orifice may be shaped in other suitable manners with efficacy, as needed or desired, for example, oval, ellipsoidal, and the like, or some other shape configured and adapted for effective aqueous transmission enabling to conform to the shape and size of the existing outflow pathways. The stent implant 31a may comprise one or more outlet ports 36 at the outlet section 33 to allow adequate outflow of aqueous, as needed or desired.

As best seen in FIG. 6, aqueous from the anterior chamber 20 enters the lumen 34 through orifice 35 and passes through the stent in a direction generally indicated by arrow 40 and exits through the lumen orifice 36 into Schlemm's canal 22 in a direction generally indicated by arrows 49. Advantageously, the stent implant 31a assists in facilitating the outflow of aqueous in an outward direction 40 through the stent 31a and into Schlemm's canal 22 and subsequently into the aqueous collectors and the aqueous veins 23 (see FIG. 2) so that the intraocular pressure (IOP) is balanced.

Preferably, in accordance with some embodiments, the entire exposed surface of the stent 31 is biocompatible and tissue compatible so that the interaction/irritation between its surface and the surrounding tissue or aqueous is minimized. In modified embodiments, selected portions or surfaces of the stent 31 may comprise a biocompatible and/or tissue compatible material, as needed or desired.

As the skilled artisan will readily appreciate, the stent implant 31 of embodiments of the invention may be dimensioned in a wide variety of manners. In an exemplary embodiment, the stent implant 31 has a length between about 0.3 millimeters (mm) to about over 1 centimeter (cm), depending on the body cavity where the stent implant is to be implanted. The outside or outer diameter of the stent implant 31 may range from about 30 micrometers or microns (μm) to about 560 μm or more. The lumen diameter is preferably in the range between about 10 μm to about 150 μm or larger. In other embodiments, the stent implant 31 may be dimensioned in modified manners with efficacy, as required or desired, giving due consideration to the goals of achieving one or more of the benefits and advantages as taught or suggested herein.

In some embodiments, the stent implant 31 comprises a biocompatible material, such as a medical grade silicone, for example, the material sold under the trademark Silastic®, which is available from Dow Corning Corporation of Midland, Mich., or polyurethane, which is sold under the trademark Pellethane®, which is also available from Dow Corning Corporation. In other embodiments, other biocompatible materials (biomaterials) may be used, such as polyvinyl alcohol, polyvinyl pyrolidone, collagen, heparinized collagen, tetrafluoroethylene, fluorinated polymer, fluorinated elastomer, flexible fused silica, polyolefin, polyester, polysilicon, stainless steel, Nitinol, titanium, a mixture of biocompatible materials, combinations thereof, and the like. In further embodiments, a composite biocompatible material may be utilized by surface coating the above-mentioned biomaterial, wherein the coating material may be selected from the group comprising polytetrafluoroethylene (PTFE), polyimide, hydrogel, heparin, therapeutic drugs, combinations thereof, and the like.

In some embodiments, the material for the stent 31 may be selected from the group comprising one or more of a porous material, a semi-rigid material, a soft material, a hydrophilic material, a hydrophobic material, a hydrogel, an elastic material, combinations thereof, and the like. The trabecular stent 31, particularly the porous stent, may have high water affinity that is hydrophilic and tissue compatible.

In some embodiments, one or more suitable drugs may be coated or loaded onto the trabecular stent 31 or an implant in the anterior/posterior chamber and slowly released to the surrounding tissue effective to treat glaucoma and/or other ophthalmology abnormalities. As is well known in the art, a device coated or loaded with a slow-release drug can have prolonged effects on local tissue surrounding the device. The slow-release delivery can be designed such that an effective amount of drug, including mitochondria stimulating agent, is released over a desired duration. The term “drug”, as used herein, is generally defined as, but not limited to, any therapeutic or active substances that can stop, mitigate, slow-down or reverse undesired disease processes.

In some embodiments, the device 31 comprises a biodegradable (also including bioerodible) material admixed with a drug for drug slow-release into ocular tissues. In other embodiments, polymer films may function as drug containing release devices whereby the polymer films may be coupled or secured to the device 31. The polymer films may be designed to permit the controlled release of the drug, including mitochondria stimulating agent, at a chosen rate and for a selected duration, which may also be episodic or periodic. Such polymer films may be synthesized such that the drug is bound to the surface or resides within the film so that the drug is relatively protected from enzymatic attack. The polymer films may also be efficaciously modified to alter their hydrophilicity, hydrophobicity and vulnerability to platelet adhesion and enzymatic attack, as needed or desired.

The polymer in accordance with embodiments of the invention should be biocompatible, for example a polymeric material that, in the amounts employed, is non-toxic and chemically inert as well as substantially non-immunogenic and non-inflammatory. Suitable polymeric materials can include, but are not limited to, polycaprolactone (PCL), poly-D,L-lactic acid (DL-PLA), poly-L-lactic acid (L-PLA), poly(lactide-co-glycolide), poly(hydroxybutyrate), poly(hydroxybutyrate-co-valerate), polydioxanone, polyorthoester, polyanhydride, poly(glycolic acid), poly(glycolic acid-cotrimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, polyiminocarbonates, and aliphatic polycarbonates, fibrin, fibrinogen, cellulose, starch, collagen, polyurethane, polyethylene, polyethylene terephthalate, ethylene vinyl acetate, ethylene vinyl alcohol, silicone, polyethylene oxide, polybutylene terephthalate (PBT)-co-PEG, PCL-co-PEG, PLA-co-PEG, polyacrylates, polyoxaesters, polyvinyl pyrrolidone (PVP), polyacrylamide (PAAm), and combinations thereof.

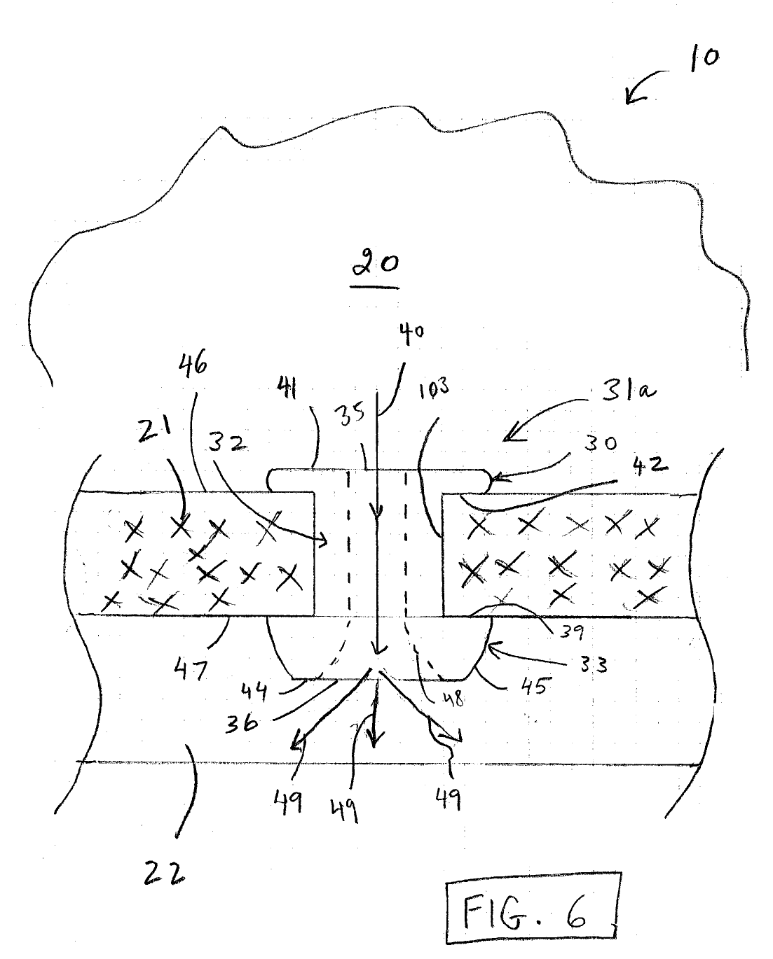

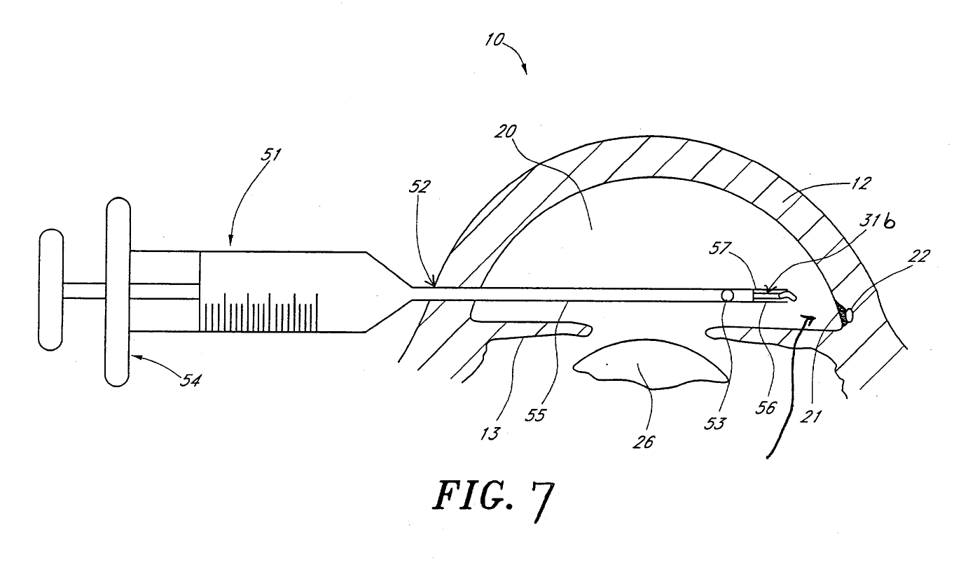

FIG. 7 illustrates one preferred method for placing a stent implant or other suitable stent device or implant at an implant site within an eye. In the illustrated embodiment, a stent implant 31b is shown though any of the other stents 31 may efficaciously be placed utilizing the method of FIG. 7.

Referring in particular to FIG. 7, an irrigating knife or applicator 51 generally comprises a syringe portion 54 and a cannula portion 55. The distal section of the cannula portion 55 may have at least one irrigating hole 53 and a distal space 56 for holding a stent implant, such as the stent implant 31b, as shown in FIG. 7. The proximal end 57 of the lumen of the distal space 56 may be sealed from the remaining lumen of the cannula portion 55 to prevent undesirable fluid leakage.

For guiding and/or positioning the stent 31 to and/or within the hole or opening or a virtual opening through the trabecular meshwork 21 (the hole or opening or a virtual opening through the trabecular meshwork is collectively also referred to as “access” herein), the stent 31 may be advanced over a guidewire, a fiberoptic (retrograde), and other suitable means. In other embodiments, the stent 31 is directly placed on the delivery applicator and advanced to the implant site, wherein the delivery applicator holds the stent 31 securely during the delivery stage and releases it during the deployment stage after an opening or “access” is created using the “trabecular microsurgery means” as taught or suggested herein.

In one preferred embodiment of the trabecular meshwork surgery, the patient is placed in the supine position, prepped, draped and administered anesthesia. In one embodiment, a small (less than or about 1 mm) self-sealing incision 52 (FIG. 7) is made in the cornea 12. Through the cornea 12 opposite the stent placement site (generally designated by the reference numeral 50 in FIG. 7), an incision or opening 103 (FIG. 6) is made in the trabecular meshwork 21 with an irrigating knife.

Still referring in particular to FIG. 7, the stent 31b is then advanced through the corneal incision 52 across the anterior chamber 20 held in the irrigating applicator 51 under gonioscopic (lens) and/or endoscopic guidance. An improved endoscope with connection to a microscope eyepiece, having features and advantages in accordance with some embodiments, is discussed in more detail below. The stent 31b is suitably positioned and implanted at the desired stent placement site 50. The applicator 51 (without the stent) is withdrawn and the surgery concluded. The irrigating knife may be within a size range of about 20 to about 40 gauges, preferably about 30 gauges.

In accordance with further embodiments, FIGS. 6 and 7 illustrate a method for increasing aqueous humor outflow in an eye 10 of a patient to reduce the intraocular pressure (IOP) therein. The method generally comprises (a) creating the opening or access 103 in the trabecular meshwork 21 by piercing means of the applicator 51 or piercing means of the stent 31, wherein the trabecular meshwork 21 comprises an interior side 46 (FIG. 6) and an exterior side 47 (FIG. 6); (b) inserting the stent device 31 into the opening or access 103 in the trabecular meshwork 21; (c) transporting the aqueous humor by the stent device 31 to bypass the trabecular meshwork 21 at the level of the trabecular meshwork from the interior side 46, facing the anterior chamber 20, to the exterior side 47, facing Schlemm's canal 22, of the trabecular meshwork 21; and/or (d) releasing, delivering or providing one or more mitochondria stimulating agents into the trabecular meshwork 21 or the outflow pathways. The outflow pathways may include, but are not limited to, Schlemm's canal, aqueous collector channels, aqueous veins, and episcleral veins, as described above.

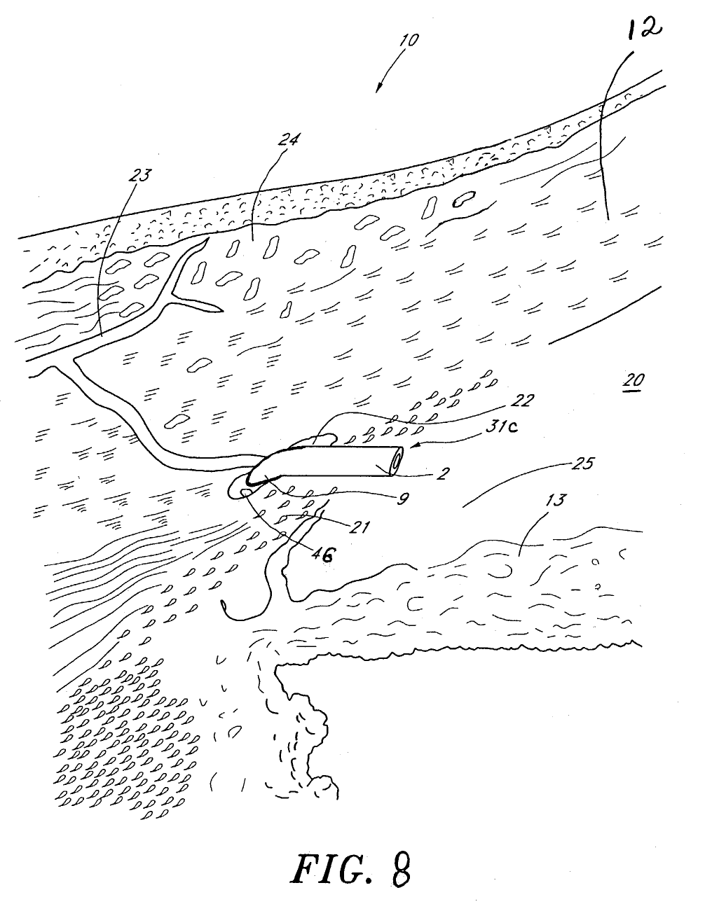

In accordance with some embodiments, FIG. 8 generally illustrates the use of a trabecular stenting device 31 for establishing an outflow pathway passing from the anterior chamber 20 through the trabecular meshwork 21 to Schlemm's canal 22. In the illustrated embodiment, a stent implant 31c is shown though any of the other stents 31 may efficaciously be used in conjunction with the method of FIG. 8. The stent 31c is positioned within the trabecular meshwork 21 of the eye 10.

As illustrated in FIG. 8, an outlet section 9 of the device 31c has been inserted in substantially its entirety into the opening in the trabecular meshwork 21. An inlet section 2 of the device 31c is exposed to the anterior chamber 20, while the outlet section 9 is positioned near the interior surface or side 46 of Schlemm's canal 22. In other embodiments, the outlet section 9 may advantageously be placed into fluid communication with other natural outflow pathways, such as, but not limited to, aqueous collector channels, aqueous veins, and episcleral veins, as described above. In some embodiments, one or more mitochondria stimulating agents are released, delivered or provided to the trabecular meshwork 21 and/or other outflow pathways.

Accordingly, some embodiments of the invention provide a system and method for stimulating mitochondria so as to mitigate apoptotic degradation of optic nerve cells for neural protection. More particularly, in some embodiments, a mitochondria stimulating drug or agent is incorporated or loaded into or onto a stent implant (such as the stent device 31) for drug slow release in an eye.

Microscope-Eyepiece and Endoscope Interface

For trabecular stent implantation, a microscope along with an endoscope is generally needed for visualization. The use of one device, for example, the microscope, followed by the use of the other, that is, the endoscope, does not facilitate accurate determinations or orderly procedures which, of course, are desired. When the microscope and endoscope are used in sequence, the surgeon must alternately look through the oculars of each device. But, undesirably, this is not easily done and does not enable certain operations to be carried out or results in time-consuming procedures. Disadvantageously, this can not only add to the cost but may also cause patient discomfort due to the length of the surgical procedure.

Some aspects of the invention provide a microscope eyepiece (or ocular) interface for an endoscope. Advantageously, this allows an individual looking through the microscope to have a combined view of the microscope image and the endoscope image. In some embodiments, such an endoscope connection to a microscope via an eyepiece assists in implantation of a glaucoma stent or other opthalmologic stent or device within an eye. In other embodiments, other types of surgical procedures may efficaciously utilize this endoscope-eyepiece interface, as needed or desired.

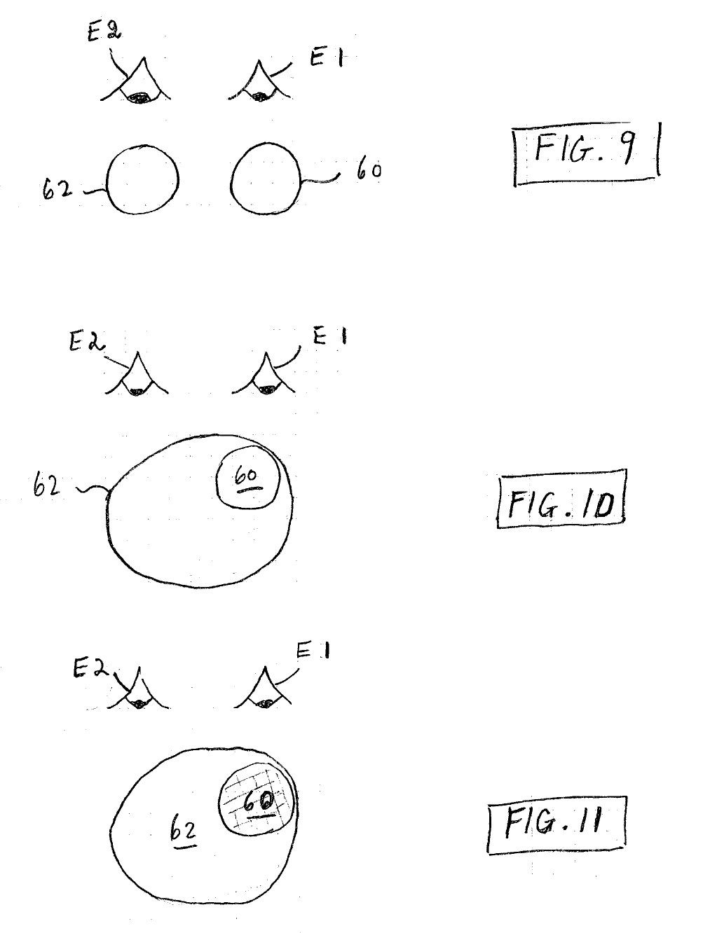

In the case of a stereomicroscope or binocular microscope, in some embodiments and as illustrated in FIG. 9, one eye E1 could view an endoscope image 60 while the other eye E2 views a microscope image 62. In other embodiments, and as illustrated in FIG. 10, the endoscope image 60 could occupy a portion of the visual field of the eyepieces and thus be adjacent to the microscope image 62. In further embodiments, and as illustrated in FIG. 11, the endoscope image 60 could be overlaid on the microscope image 62 seen through the microscope eyepieces. The overlay may be a partial overlay as shown in FIG. 11 or a complete overlay, as needed or desired.

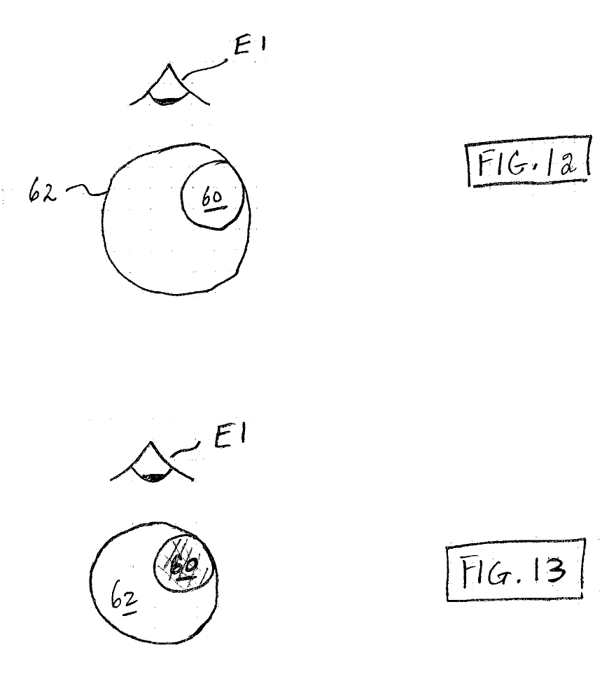

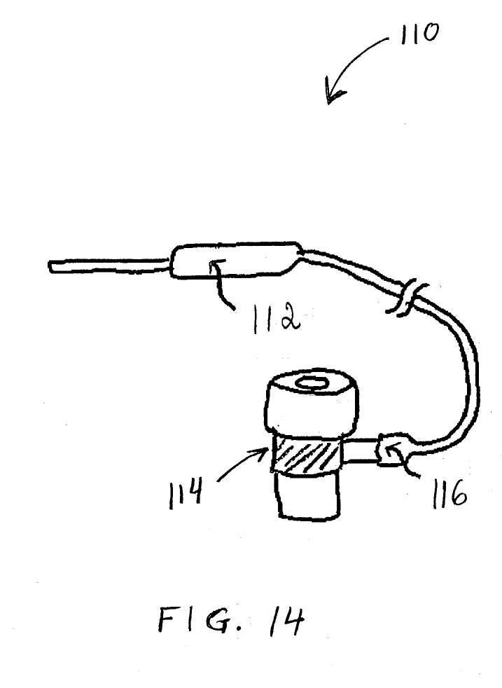

In the case of a monocular microscope, in some embodiments and as illustrated in FIG. 12, the endoscope image 60 could occupy a portion of the visual field of the eyepiece and thus be adjacent to the microscope image 62. In other embodiments, and as illustrated in FIG. 13, the endoscope image 60 could be overlaid on the microscope image 62 seen through the microscope eyepiece. The overlay may be a partial overlay as shown in FIG. 13 or a complete overlay, as needed or desired.

Accordingly, some aspects of the invention relate to providing a simultaneous view of the microscope field of view and the endoscope field of view to the operator or surgeon. This is particularly useful when the position of the endoscope or other instruments needs to be observed while also viewing the field through the endoscope. As discussed further below, an added advantage is that the custom eyepiece of embodiments of the invention can readily be inserted into a standard microscope eye tube or mounted thereon, thus desirably eliminating the need to modify or replace the microscope body. This retrofit connection saves on cost and also adds to the versatility and utility of the device.

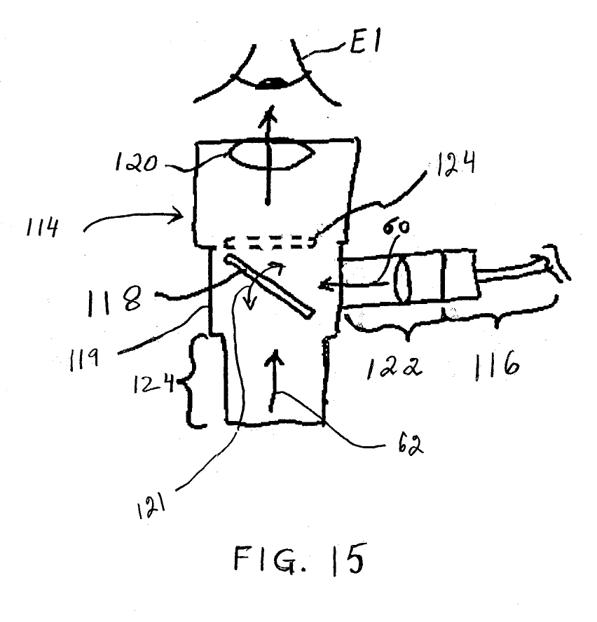

FIG. 14 depicts an optical assembly, system or apparatus 110 which facilitates simultaneous viewing of endoscopic and operating microscopic images. The assembly 110 generally comprises an endoscope 112 interfaced to a custom eyepiece or ocular 114 (described further below) utilizing a connector 116. Preferably, the endoscope 112 comprises a fiber bundle endoscope though other suitable endoscopes may be efficaciously utilized, as needed or desired. In one aspect, the fiber bundle endoscope 112 collects, captures or retrieves an image and delivers the image to the connector 116 that interfaces to the custom microscope eyepiece 114.

FIG. 15 is a schematic depiction of some of the optical details of the interconnection between the endoscope connector 116 and the custom eyepiece 114. The image 60 from the endoscope 112 is delivered via a complete or partially reflecting surface 118 through the eyepiece lens 120 to the viewer's eye E1. The image 62 from the microscope is combined with the image 60 from the endoscope 112 to a degree substantially determined by the reflectance of the reflecting surface 118. The reflecting surface 118 is housed within a tubular section or portion 119 of the eyepiece or ocular 114. In some embodiments, the reflecting surface or mirror 118 may be movable in/out of the field of view as generally depicted by the arrow(s) 121.

Referring in particular to FIG. 15, in some embodiments, suitable expansion optics 122 are used to expand the tiny image on the end of the fiber bundle at the connector 116 to make the endoscope image 60 comparable to the microscope image 62 if an overlay (FIGS. 11 and 13) or side-by-side view (FIGS. 10 and 12) is desired. Of course, in the case of a stereomicroscope, and as illustrated in FIG. 9, one eye E1 could view the endoscope image 60 while the other eye E2 views the microscope image 62.

Still referring in particular to FIG. 15, in other embodiments, the tiny image on the end of the fiber bundle at the connector 116 might be expanded to a smaller degree and placed on a suitable medium such as a ground glass screen 124 (shown in phantom). This may reduce the potential to include the image 62 from the microscope field and in the case of a stereomicroscope it may be desirable to use one eye E1 to view the endoscope image 60 while the other eye E2 views the microscope image 62, as illustrated in the embodiment of FIG. 9.

In the illustrated embodiment of FIG. 15, the eyepiece or ocular 114 has a reduced diameter portion or section 124 at the end opposite the lens 120. The custom eyepiece 114 fits into the microscope body of a standard stereomicroscope or monocular microscope (as discussed below) at the reduced diameter portion 124. This advantageously allows for a simple retrofit connection.

In some embodiments, the endoscope 112 (or other suitable device) is used to deliver any of the drugs as taught or suggested herein including the mitochondrial stimulating agents, compounds or drugs discussed further below. The drug(s) are delivered to a desired site within the eye to treat the medium thereof. The drugs may be administered by the endoscope 112, for example, by using an ab interno procedure.

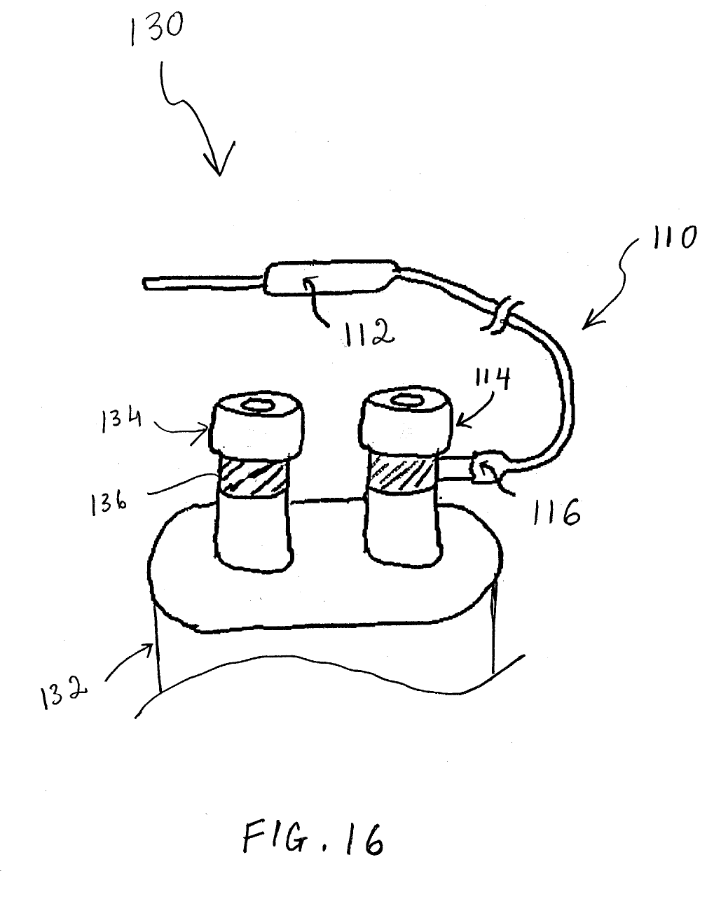

FIG. 16 shows a stereomicroscope or binocular microscope and endoscope assembly, apparatus, system or combination 130 which advantageously provides a simultaneous view of the microscope field of view and the endoscope field of view to the operator or surgeon as discussed above in connection with FIGS. 9-11. The microscope assembly 130 generally comprises a conventional or other stereomicroscope body 132, a second or left eyepiece or ocular 134 and the optical assembly 110 including the custom eyepiece or ocular 114 interfaced with the fiber bundle endoscope 112 via the connector 116.

In the illustrated embodiment of FIG. 16, to accommodate the interface optics within the tubular section 119 (see FIG. 15), the custom microscope eyepiece 114 is generally longer than a conventional eyepiece. Accordingly, in some embodiments, the left eyepiece 134 comprises a spacer tube 136 so that it is at about the same level or length as the custom eyepiece 114. The eyepieces 114, 134 are inserted into one or more tubes (not shown) on the conventional stereomicroscope body 132 to mount or fit the eyepieces 114, 134 on the conventional stereomicroscope body 132.

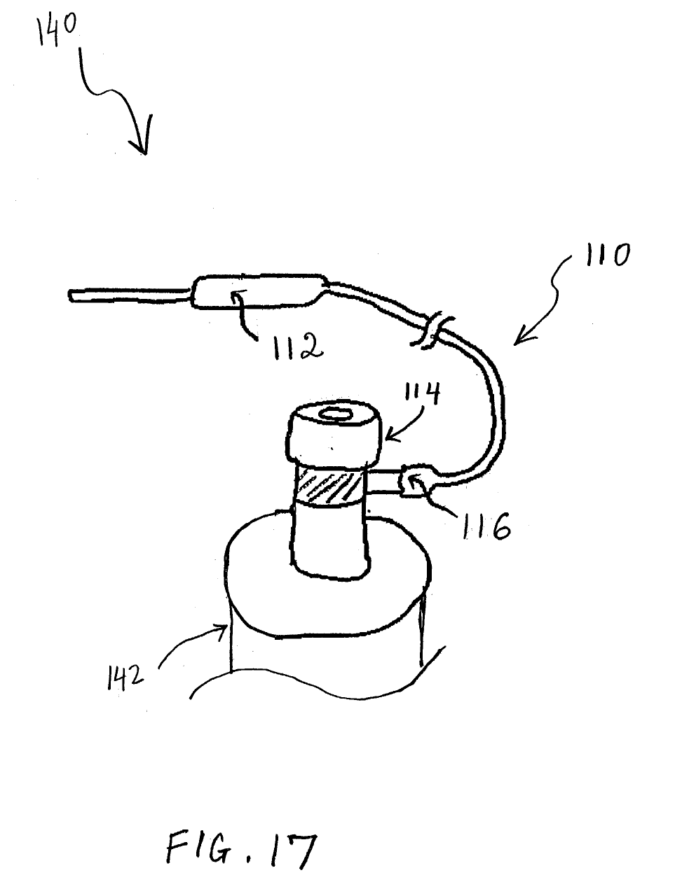

FIG. 17 shows a monocular microscope and endoscope assembly, apparatus, system or combination 140 which advantageously provides a simultaneous view of the microscope field of view and the endoscope field of view to the operator or surgeon as discussed above in connection with FIGS. 12 and 13. The microscope assembly 140 generally comprises a conventional or other monocular microscope body 142 and the optical assembly 110 including the custom eyepiece or ocular 114 interfaced with the fiber bundle endoscope 112 via the connector 116. The eyepiece 114 may be inserted into one or more tubes (not shown) on the conventional monocular microscope body 142 to mount or fit the eyepiece 114 on the conventional monocular microscope body 142.

Many conventional stereomicroscopes and monocular microscopes have an additional port for providing the microscope image viewed by the surgeon or operator to a second individual such as the surgeon's assistant. In some embodiments, as discussed below, an endoscope is interfaced at this port to provide a simultaneous view of the microscope field of view and the endoscope field of view to the operator or surgeon.

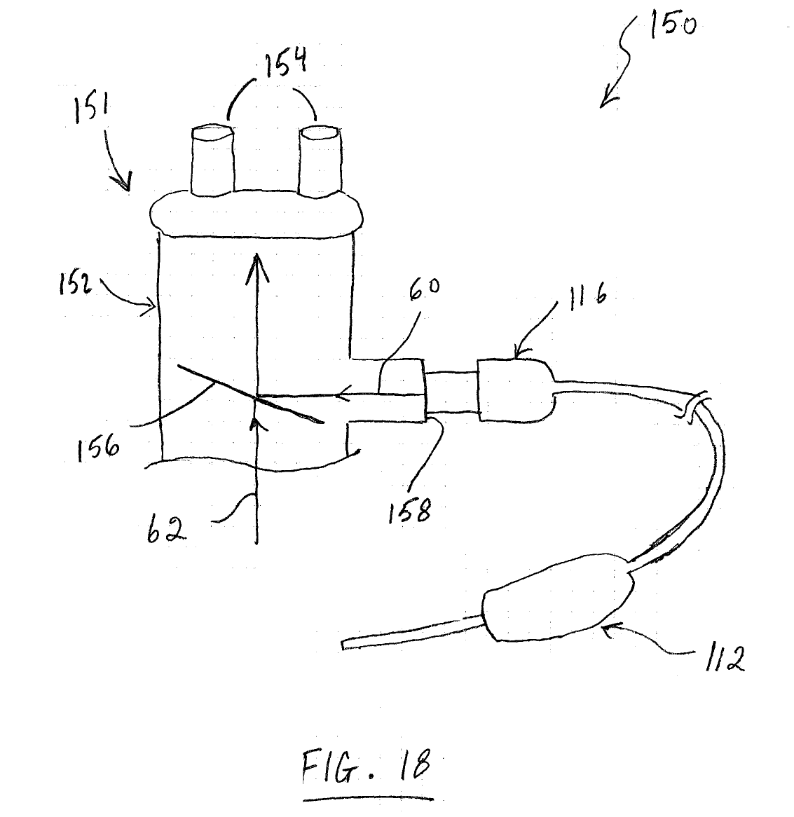

FIG. 18 shows a modified embodiment of a stereomicroscope or binocular microscope and endoscope assembly, apparatus, system or combination 150 which advantageously provides a simultaneous view of the microscope field of view and the endoscope field of view to the operator or surgeon as discussed above in connection with FIGS. 9-11. The microscope assembly 150 generally comprises a conventional or other stereomicroscope 151 interfaced with an endoscope 112 via a connector 116 through an already existing port 158 on the stereomicroscope body 152. Advantageously, such a retrofit connection between the conventional stereomicroscope 151 and endoscope 112 allows the interface to be created without the need to substantially modify or replace the microscope body 152. Desirably, this saves on cost and adds to the versatility and utility of the device.

In the illustrated embodiment of FIG. 18, a beam splitter 156 (or one or more other suitable optical elements) directs the combined (including overlaid, parallel or side-by-side) views of the microscope image 62 and endoscope image 62 to the eyepieces or oculars 154. Suitable interface and/or expansion optics may be efficaciously utilized, as needed or desired. In some embodiments, the endoscope image 60 may be placed on a suitable medium such as a ground glass screen as discussed above in connection with FIG. 15.

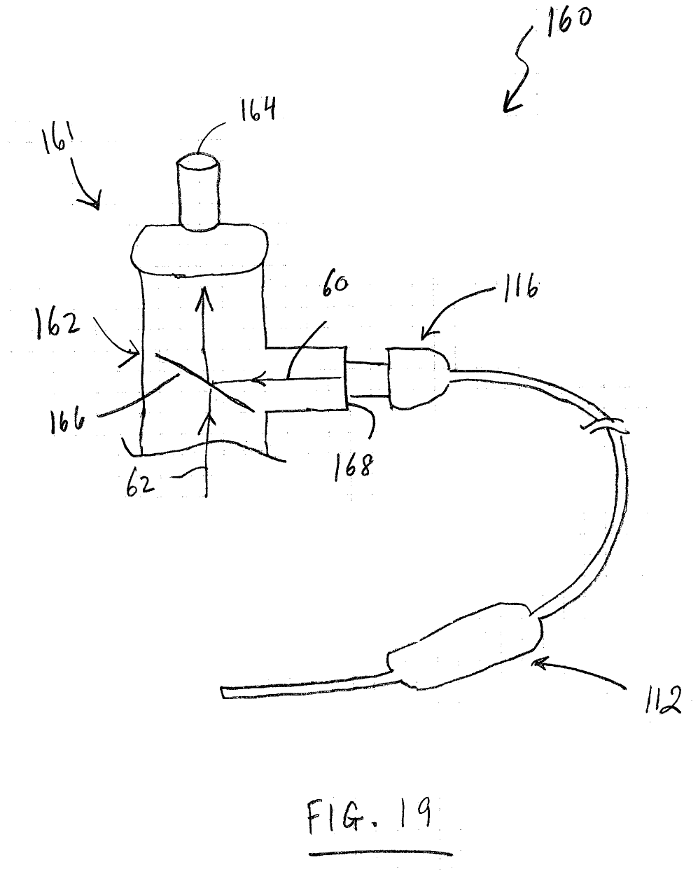

FIG. 19 shows a modified embodiment of a monocular microscope and endoscope assembly, apparatus, system or combination 160 which advantageously provides a simultaneous view of the microscope field of view and the endoscope field of view to the operator or surgeon as discussed above in connection with FIGS. 12 and 13. The microscope assembly 160 generally comprises a conventional or other monocular microscope 161 interfaced with an endoscope 112 via a connector 116 through an already existing port 168 on the monocular microscope body 162. Advantageously, such a retrofit connection between the conventional monocular microscope 161 and endoscope 112 allows the interface to be created without the need to substantially modify or replace the microscope body 162. Desirably, this saves on cost and adds to the versatility and utility of the device.

In the illustrated embodiment of FIG. 19, a beam splitter 166 (or one or more other suitable optical elements) directs the combined (including overlaid or side-by-side) views of the microscope image 62 and endoscope image 62 to the eyepiece or ocular 164. Suitable interface and/or expansion optics may be efficaciously utilized, as needed or desired. In some embodiments, the endoscope image 60 may be placed on a suitable medium such as a ground glass screen as discussed above in connection with FIG. 15.

Mitochondrial Stimulating Therapy

In a normal eye, aqueous humor is produced in the ciliary body, flows between the lens and the iris into the anterior chamber, and the majority passes through the trabecular meshwork (TM) to the episcleral veins. The aqueous humor is an ultrafiltrate containing salts and nutrients that bathes the lens and cornea and removes metabolic waste products. In primary open angle glaucoma (POAG), the aqueous outflow capacity is diminished and the number of juxtacanalicular endothelial cells that are involved in the egress of aqueous in Schlemm's canal is reduced compared to normals (Grierson et al., Exp Eye Res, 1984; 39(4):505-512). These reductions result in a secondary elevation of intraocular pressure (IOP) that leads to eventual blindness through the death of neurons in the optic nerve and loss of nerve fiber layer in the retina.

At present, a number of theories or hypotheses exist that attempt to explain how the trabecular meshwork facilitates the flow of aqueous from the anterior chamber of the eye to Schlemm's canal and the episcleral venous system. These can be summarized as: passive sieve, active/passive vacuole transport, and passive pump. The descriptions provided below summarize these various theories and discuss the relevance of mitochondria to each of these viewpoints. Understanding the functionality of the meshwork and its diseased malfunction remain active areas of current research.

Mitochondria are the main energy source in cells of higher organisms, and these cells provide direct or indirect biochemical regulation of a wide variety of cellular respiratory, oxidative and metabolic processes. These include electron transport chain activity, which drives oxidative phosphorylation to produce metabolic energy in the form of adenosine triphosphate (ATP). In metabolic processes, mitochondria are also involved in the genetically programmed cell death known as apoptosis. Defective or dysfunctional mitochondrial activity may alternately result in the generation of highly reactive free radicals that have the potential of damaging cells and tissues. It was thought that mitochondrial participation in the apoptotic cascade is believed to be a key event in the pathogenesis of neuronal death.

Passive Sieve:

This theory describes the trabecular meshwork as a sieve-like structure that starts out coarse and becomes finer as it progresses through the meshwork from the anterior chamber toward Schlemm's canal. The sieve-like structure prevents anterior-chamber particles (e.g. lens particles from pseudoexfoliation glaucoma and iris particles from pigmentary glaucoma) from passing into Schlemm's canal and the collector channels causing potential occlusion of outflow in these downstream structures. The “sieve” also prevents reflux of blood cells into the anterior chamber during periods of reversed flow caused by opening/depressurizing the eye or by occluding an episcleral vein.

In a glaucomatous individual the flow-resistance of this sieve-like structure increases thus causing an increase in the intraocular pressure (IOP). The increase in resistance is believed to be caused by abnormal metabolism within the trabecular cells that leads to a buildup of extra-cellular matrix material that impedes the flow of aqueous through the meshwork. Additionally, the meshwork cells are believed to be phagocytotic and that this phagocytotic capacity decreases in glaucomatous individuals (Matsumoto et al. Ophthalmologica 1997; 211:147-152).

The mitochondria within these cells provide the energy source for the cells; adjustment of this energy source with mitochondrial drugs could help to alleviate the extra-cellular buildup and/or increase the phagocytotic activity, thus reducing the outflow resistance of the meshwork.

Active/Passive Vacuole Transport:

This theory describes the trabecular meshwork as a sieve-like structure with a juxtacanalicular layer that modulates aqueous flow into Schlemm's canal through pore-like openings (Shields, Williams & Wilkins, Baltimore 1982). Large aqueous-filled invaginations are engulfed on the meshwork side of the layer and move across the juxtacanalicular layer to the inner wall of Schlemm's canal where they open via small pores to deliver the aqueous to Schlemm's canal. Competing theories classify this process as either active or passive. Researchers have shown that the pore density and number of vacuoles increases with intraocular pressure (IOP).

In a glaucomatous individual this transport mechanism is slowed such that the IOP increases in response to the slower egress of fluid from the eye. This could be a passive response to a buildup of extra-cellular matrix if this is a passive mechanism, or could be related to a decreased availability of necessary energy for the process.

The mitochondria within these cells provide the energy source for the cells; adjustment of this energy source with mitochondrial drugs should help to enhance this outflow pathway so as to improve aqueous egress and thus reduce IOP.

Passive/Active Pump:

This theory describes the trabecular meshwork as having tube-like extensions (Johnstone tubules) that extend across the Schlemm's canal and direct aqueous toward collector channel openings (Johnstone et al., AGS 2002 Meeting 2/28-3/3/02, Puerto Rico, paper #18). The meshwork is believed to expand and compress in response to the ocular pulse thus promoting the flow of aqueous from the meshwork to the collector channels. The process may be passive or it may have active elements or processes that respond to changes in intraocular pressure (IOP) to adjust the pumping volume of the meshwork and tubules.

In a glaucomatous individual a passive pumping process may be impeded by the presence of extra-cellular matrix that could be alleviated by mitochondrial drugs (as described above). Alternatively, a pumping process with active energy input may benefit from active manipulation of the mitochondrial energy source using mitochondrial therapy.

The dysequilibrium or imbalance between formation and outflow of aqueous humor underlies primary open angle glaucoma (POAG) and both are heavily energy-dependent processes. The primary defect is an increased resistance to outflow, rather than an over production of aqueous.

Therefore, therapeutic strategies focused on improving mitochondrial integrity and ATP production in the glaucomatous eye may show efficacy by preventing decreases in outflow and by preventing secondary retinal cell apoptosis. It may be possible, through mitochondrial rescue and ATP production boosting, to maintain normal IOPs in early POAG patients. In addition, since the target site is the trabecular meshwork, it may be possible to develop a topical medication that would greatly decrease the potential for side effects compared to a systemic drug. In another aspect, the drug slow release therapy to target tissue may allow the use of a drug-coated implant, including a mitochondrial stimulating agent, in an eye.

In a recently reported study (Putney et al., Am J Physiol, 1999; 277:C373-383), human trabecular meshwork cells were harvested from eye-bank donor rims and cultured to explore the affect of intracellular Cl⁻ on Na⁺—K⁺—Cl⁻ cotransport activity. This cotransporter activity was previously found to maintain steady-state cell volume most likely by offsetting ion efflux pathways such as K⁺ and Cl⁻ channels and/or K⁺—Cl⁻ cotransport (Parker, in Cellular and Molecular Physiology of Cell Volume Regulation, edited by K. Strange, Boca Raton, Fla.: CRC, p. 311-321 (1994)). Reduction in the size of the cells, increases the intercellular space and reduces the resistance to outflow in the trabecular meshwork (TM).

This phenomenon has been assessed from a cellular energy standpoint of cells in the juxtacanalicular endothelial lining. The cells, due to degradation in the performance of cellular mitochondria do not produce sufficient energy to enable the adequate active transport of aqueous (either across the cell to move the fluid from one side to the other side or into and out of the cell to change its size). This decrease in active transport leads to a buildup of fluid pressure in the eye (symptom of glaucoma) that results in damage to the retinal neurons.

Treatment of these mitochondria with appropriate compounds that improve their performance or stimulate their function may improve the active transport of fluid and thus alleviate the buildup of aqueous in the eye. Reduction of the IOP in glaucomatous individuals is widely accepted as a means of preserving the vitality of the optic nerve.

Previous research in the area of mitochondria and glaucoma exists. A monoamine oxidase inhibitor, deprenyl, that has been used in the treatment of Parkinson's disease may play a role in reducing neuronal apoptosis in glaucoma (Tatton, Eur J Ophthalmol, 1999;9(suppl 1):S22-29). Tatton in U.S. Pat. No. 5,981,598 further discloses that the primary metabolite of deprenyl, desmethyldeprenyl (DES), is involved in the maintenance of the mitochondrial membrane and prevents apoptotic degradation. A continuation of this work was recently reported by Tatton, et al. (Survey of Ophthalmol 2001; 45(S3):S277-283). Another interesting review article by Nickells espouses to the future design of new treatments for glaucoma provided a better understanding of apoptosis can be achieved (Nickells, Survey of Ophthalmol 1999; 43 (S1):S151-161).

Prevention or slowing of apoptotic degradation of optic nerve cells provides a form of neural protection that will aid in the preservation of sight for individuals suffering from either “low tension” glaucoma or hypertensive glaucoma. It is one aspect of some embodiments of the invention to provide a method for administering appropriate compounds at an amount effective to energize the mitochondria in the neurons and aid the cells by enabling them to better remove substances that lead to their apoptotic degradation.

It is another aspect of some embodiments of the invention to provide a method for administering appropriate compounds at an amount effective to energize mitochondria in a neuron enabling the neuron to better remove apoptotic waste so as to revive or rejuvenate the neuron. The method further comprises loading the compounds onto or within an ophthalmologic implant, wherein the ophthalmologic implant is a trabecular stent implanted in trabecular meshwork of an eye. The ophthalmologic implant may also been implanted in an anterior or posterior chamber of an eye.

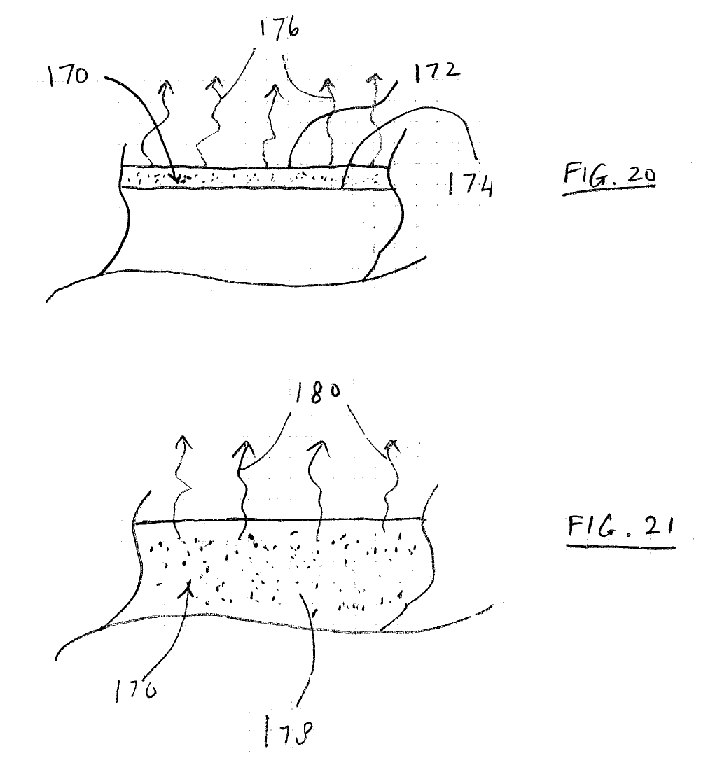

In some embodiments, and as illustrated in FIG. 20, the drug(s) or compound(s) 170 are provided in the form of a coating or film 172 on the surface 174 of the implant or device for timed release into or onto the desired site as generally indicated by arrows 176. In other embodiments, and as illustrated in FIG. 21, the drug(s) or compound(s) 170 are provided within the material 178 of the implant or device for timed release into or onto the desired site as generally indicated by arrows 180. These embodiments may also be efficaciously combined in a desired configuration or pattern to release drug from the surface and within the material of the implant, as needed or desired.

It is a further aspect of some embodiments of the invention to activate mitochondria of ophthalmology cells for enhanced aqueous transmission comprising an energy source with activating energy effective for activating mitochondria. The energy source may be a physical source selected from a group comprising ultrasound ablation energy, ultrasonic vibrational energy, microwave energy, optical light energy, laser energy, electromagnetic energy, and combination thereof. Suitable transducers and the like may be used to provide this energy. The mode of energy stimulation may be continuous, intermittent, programmed, or combinations thereof.

Some embodiments provide a method of treating mitochondria in a cell of a glaucoma patient. The method generally comprises stimulating mitochondria of the cell with an energy source sufficient to increase cellular energy production.

In some aspects of the invention, the energy is provided by a mitochondrial stimulating agent. In some embodiments, the mitochondrial stimulating agent comprises a monoamine oxidase inhibitor such as a deprenyl compound.

From the foregoing description, it will be appreciated that a novel approach for treating glaucoma and/or elevated intraocular pressure (IOP) has been disclosed. While the components, techniques and aspects of the invention have been described with a certain degree of particularity, it is manifest that many changes may be made in the specific designs, constructions and methodology herein above described without departing from the spirit and scope of this disclosure.

Although preferred embodiments of the invention have been described in detail, including ab interno procedures and devices thereof, certain variations and modifications will be apparent to those skilled in the art, including embodiments that do not provide all of the features and benefits described herein. Accordingly, the scope of the invention is not to be limited by the illustrations or the foregoing descriptions thereof, but rather solely by reference to the appended claims.

Various modifications and applications of the invention may occur to those who are skilled in the art, without departing from the true spirit or scope of the invention. It should be understood that the invention is not limited to the embodiments set forth herein for purposes of exemplification, but is to be defined only by a fair reading of the, appended claims, including the full range of equivalency to which each element thereof is entitled.

What is claimed is:

1. A method of treating mitochondria in a cell of a glaucoma patient comprising stimulating mitochondria of the cell with an energy source sufficient to increase cellular energy production.

2. The method of claim 1, wherein the energy is selected from the group consisting of ultrasound energy, microwave energy, optical light energy, laser energy, and electromagnetic energy.

3. The method of claim 2, wherein a mode of delivering energy is selected from the group consisting of continuous, intermittent, and programmed.

4. The method of claim 1, wherein the energy is provided by a mitochondrial stimulating agent.

5. The method of claim 4, wherein the mitochondrial stimulating agent is a monoamine oxidase inhibitor.

6. The method of claim 5, wherein the monoamine oxidase inhibitor is a deprenyl compound.

7. The method of claim 4, wherein the mitochondrial stimulating agent is loaded onto or within an ophthalmologic implant.

8. The method of claim 7, wherein the ophthalmologic implant is a trabecular stent that is configured to be implantable in a trabecular meshwork of the patient.

9. The method of claim 7, wherein the ophthalmologic implant is implanted in a posterior chamber of the patient's eye.

10. The method of claim 7, wherein the ophthalmologic implant is implanted in an anterior chamber of the patient's eye.
